(19) United States
(12) Patent Application Publication
(10) Pub. No.: US 2014/0173888 A1
(43) Pub. Date: Jun. 26, 2014

(54) Method and Apparatus for Coupling Micro-Components Together Using a Micro-Clip

(71) Applicant: Volcano Corporation, San Diego, CA (US)

(72) Inventor: Michael Reiter, San Diego, CA (US)

(73) Assignee: Volcano Corporation, San Diego, CA (US)

(21) Appl. No.: 14/103,271

(22) Filed: Dec. 11, 2013

Related U.S. Application Data
(60) Provisional application No. 61/745,442, filed on Dec. 21, 2012.

(51) Int. Cl. F16B 2/22; A61B 8/00

(52) U.S. Cl. CPC F16B 2/22; A61B 8/4483
USPC 29/594; 24/555

(57) ABSTRACT

The present disclosure provides a micro-structure for coupling a first micro-component with a second micro-component. The micro-structure includes a center portion. The micro-structure also includes a first pair of arms connected to the center portion and extending away from the center portion in a first direction. The first pair of arms are separated by a first gap that is approximately equal to, or less than, a thickness of the first micro-component. The micro-structure also includes a second pair of arms connected to the center portion and extending away from the center portion in a second direction different from the first direction. The second pair of arms are separated by a second gap that is approximately equal to, or less than, a thickness of the second micro-component.

### CROSS-REFERENCE TO RELATED APPLICATIONS

This application claims priority to Provisional Patent Application No. 61/745,442, filed Dec. 21, 2012, and entitled “Method and Apparatus for Coupling Micro-Components Together Using a Micro-Clip,” the disclosure of which is hereby incorporated by reference in its entirety.

### TECHNICAL FIELD

The present disclosure relates generally to ultrasound imaging, and in particular, to a method and apparatus for providing electrical connections to a miniature ultrasound transducer, such as a piezoelectric micromachined ultrasound transducer (PMUT), used for intravascular imaging.

### BACKGROUND

Intravascular ultrasound (IVUS) imaging is widely used in interventional cardiology as a diagnostic tool for assessing a vessel, such as an artery, within the human body to determine the need for treatment, to guide intervention, and/or to assess its effectiveness. An IVUS imaging system uses ultrasound echoes to form a cross-sectional image of the vessel of interest. Typically, IVUS imaging uses a transducer on an IVUS catheter that both emits ultrasound signals (waves) and receives the reflected ultrasound signals. The emitted ultrasound signals (often referred to as ultrasound pulses) pass easily through most tissues and blood, but they are partially reflected by discontinuities arising from tissue structures (such as the various layers of the vessel wall), red blood cells, and other features of interest. The IVUS imaging system, which is connected to the IVUS catheter by way of a patient interface module, processes the received ultrasound signals (often referred to as ultrasound echoes) to produce a cross-sectional image of the vessel where the IVUS catheter is located.

IVUS catheters typically employ one or more transducers to transmit ultrasound signals and receive reflected ultrasound signals. However, conventional methods and apparatuses for providing electrical connections may not be optimized. For example, wire bonding is typically employed to provide electrical connections to an ultrasonic transducer. To perform wire bonding, an expensive wire bonding machine needs to be deployed, which also requires an operator who is specifically trained in its use. Wire bonding also requires in most cases a significant amount of thermal input to the bonded substrate, which may result in thermal degradation of the component. In addition, wire bonding requires a rather tall “service loop”, which increases the height of the electrical connection.

Therefore, while conventional methods and apparatuses for providing electrical connections to transducers are generally adequate for their intended purposes, they have not been entirely satisfactory in every aspect.

### SUMMARY

The present disclosure involves a micro-clip. The micro-clip is a miniature clip that has two pairs of arms that face opposite directions. One pair of arms of the clip is used to clamp to a miniature ultrasound transducer. The other pair of arms of the clip is used to clamp to an Integrated Circuit (IC) chip. Therefore, the micro-clip establishes electrical communication between the miniature ultrasound transducer and the IC chip, such that the IC chip can control or otherwise interact with the miniature ultrasound transducer.

One aspect of the present disclosure involves a micro-structure for coupling a first micro-component with a second micro-component. The micro-structure includes: a center portion; a first pair of arms connected to the center portion and extending away from the center portion in a first direction, the first pair of arms being separated by a first gap that is approximately equal to, or less than, a thickness of the first micro-component; and a second pair of arms connected to the center portion and extending away from the center portion in a second direction different from the first direction, the second pair of arms being separated by a second gap that is approximately equal to, or less than, a thickness of the second micro-component.

Another aspect of the present disclosure involves an assembly for an ultrasound imaging device. The assembly includes: a first micro-component having an upper surface and a lower surface, wherein the first micro-component includes a miniature ultrasound transducer; a second micro-component having an upper surface and a lower surface, wherein the second micro-component includes a micro-electronic Integrated Circuit (IC) chip; and a micro-clip coupled to the first and second micro-components, wherein the micro-clip includes a first pair of arms clamped to the upper and lower surfaces of the first micro-component and a second pair of arms clamped to the upper and lower surfaces of the second micro-component.

Yet another aspect of the present disclosure includes a method of fabricating a miniature ultrasound transducer. The method includes: providing a first micro-component that includes a miniature ultrasound transducer; providing a second micro-component that includes a micro-electronic Integrated Circuit (IC) chip; and coupling, using a conductive micro-clip, the first and second micro-components together, wherein electrical connections between the miniature ultrasound transducer and the IC chip are established at least in part through the micro-clip.

Both the foregoing general description and the following detailed description are exemplary and explanatory in nature and are intended to provide an understanding of the present disclosure without limiting the scope of the present disclosure. In that regard, additional aspects, features, and advantages of the present disclosure will become apparent to one skilled in the art from the following detailed description.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is a schematic illustration of an intravascular ultrasound (IVUS) imaging system according to various aspects of the present disclosure.

FIG. 2 is a diagrammatic perspective view of a transducer assembly according to various aspects of the present disclosure.

FIG. 3 is a diagrammatic perspective view of a micro-clip for coupling components of the transducer assembly of FIG. 2 according to various aspects of the present disclosure.

FIG. 4 is a diagrammatic top view of the micro-clip of FIG. 3 according to various aspects of the present disclosure.

FIG. 5 is a diagrammatic top view of an alternative embodiment of the micro-clip of FIG. 3 according to various aspects of the present disclosure.

FIG. 6 is a flowchart illustrating a method for fabricating an ultrasound transducer according to various aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, the present disclosure provides an ultrasound imaging system described in terms of cardiovascular imaging, however, it is understood that such description is not intended to be limited to this application, and that such imaging system can be utilized for imaging throughout the body. In some embodiments, the illustrated ultrasound imaging system is a side looking intravascular imaging system, although transducers formed according to the present disclosure can be mounted in other orientations including forward looking. The imaging system is equally well suited to any application requiring imaging within a small cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

There are primarily two types of catheters in common use today: solid-state and rotational. An exemplary solid-state catheter uses an array of transducers (typically 64) distributed around a circumference of the catheter and connected to an electronic multiplexer circuit. The multiplexer circuit selects transducers from the array for transmitting ultrasound signals and receiving reflected ultrasound signals. By stepping through a sequence of transmit-receive transducer pairs, the solid-state catheter can synthesize the effect of a mechanically scanned transducer element, but without moving parts. Since there is no rotating mechanical element, the transducer array can be placed in direct contact with blood and vessel tissue with minimal risk of vessel trauma, and the solid-state scanner can be wired directly to the imaging system with a simple electrical cable and a standard detachable electrical connector.

An exemplary rotational catheter includes a single transducer located at a tip of a flexible driveshaft that spins inside a sheath inserted into the vessel of interest. The transducer is typically oriented such that the ultrasound signals propagate generally perpendicular to an axis of the catheter. In the typical rotational catheter, a fluid-filled (e.g., saline-filled) sheath protects the vessel tissue from the spinning transducer and driveshaft while permitting ultrasound signals to freely propagate from the transducer into the tissue and back. As the driveshaft rotates (for example, at 30 revolutions per second), the transducer is periodically excited with a high voltage pulse to emit a short burst of ultrasound. The ultrasound signals are emitted from the transducer, through the fluid-filled sheath and sheath wall, in a direction generally perpendicular to an axis of rotation of the driveshaft. The same transducer then listens for returning ultrasound signals reflected from various tissue structures, and the imaging system assembles a two dimensional image of the vessel cross-section from a sequence of several hundred of these ultrasound pulse/echo acquisition sequences occurring during a single revolution of the transducer.

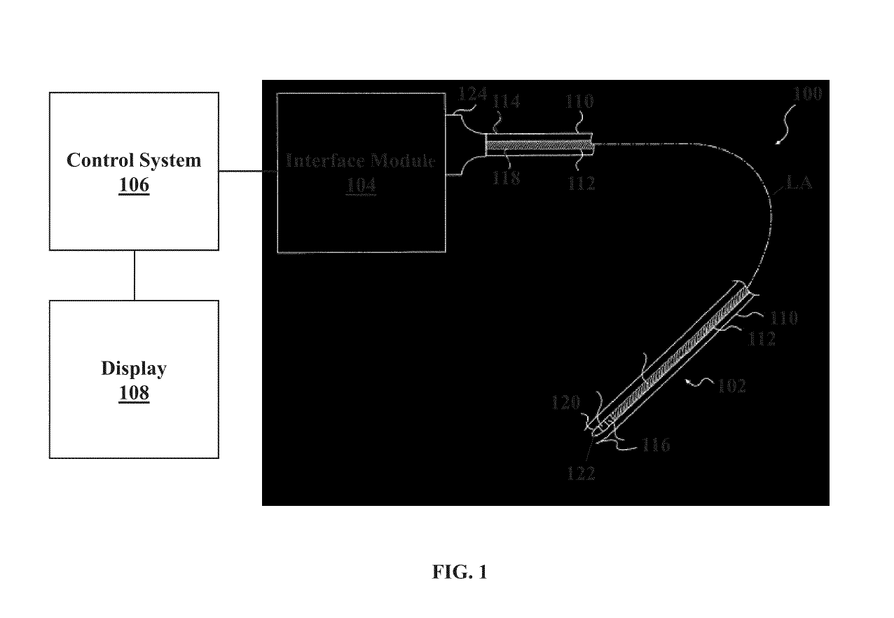

FIG. 1 is a schematic illustration of an ultrasound imaging system 100 according to various aspects of the present disclosure. In some embodiments, the ultrasound imaging system 100 includes an intravascular ultrasound imaging system (IVUS). The IVUS imaging system 100 includes an IVUS catheter 102 coupled by a patient interface module (PIM) 104 to an IVUS control system 106. The control system 106 is coupled to a monitor 108 that displays an IVUS image (such as an image generated by the IVUS system 100).

In some embodiments, the IVUS catheter 102 is a rotational IVUS catheter, which may be similar to a Revolution® Rotational IVUS Imaging Catheter available from Volcano Corporation and/or rotational IVUS catheters disclosed in U.S. Pat. No. 5,243,988 and U.S. Pat. No. 5,546,948, both of which are incorporated herein by reference in their entirety. The catheter 102 includes an elongated, flexible catheter sheath 110 (having a proximal end portion 114 and a distal end portion 116) shaped and configured for insertion into a lumen of a blood vessel (not shown). A longitudinal axis LA of the catheter 102 extends between the proximal end portion 114 and the distal end portion 116. The catheter 102 is flexible such that it can adapt to the curvature of the blood vessel during use. In that regard, the curved configuration illustrated in FIG. 1 is for exemplary purposes and in no way limits the manner in which the catheter 102 may curve in other embodiments. Generally, the catheter 102 may be configured to take on any desired straight or arcuate profile when in use.

A rotating imaging core 112 extends within the sheath 110. The imaging core 112 has a proximal end portion 118 disposed within the proximal end portion 114 of the sheath 110 and a distal end portion 120 disposed within the distal end portion 116 of the sheath 110. The distal end portion 116 of the sheath 110 and the distal end portion 120 of the imaging core 112 are inserted into the vessel of interest during operation of the IVUS imaging system 100. The usable length of the catheter 102 (for example, the portion that can be inserted into a patient, specifically the vessel of interest) can be any suitable length and can be varied depending upon the application. The proximal end portion 114 of the sheath 110 and the proximal end portion 118 of the imaging core 112 are connected to the interface module 104. The proximal end portions 114, 118 are fitted with a catheter hub 124 that is removably connected to the interface module 104. The catheter hub 124 facilitates and supports a rotational interface that provides electrical and mechanical coupling between the catheter 102 and the interface module 104.

The distal end portion 120 of the imaging core 112 includes a transducer assembly 122. The transducer assembly 122 is configured to be rotated (either by use of a motor or other rotary device or manually by hand) to obtain images of the vessel. The transducer assembly 122 can be of any suitable type for visualizing a vessel and, in particular, a stenosis in a vessel. In the depicted embodiment, the transducer assembly 122 includes a piezoelectric micromachined ultrasonic transducer (“PMUT”) transducer and associated circuitry, such as an application-specific integrated circuit (ASIC). An exemplary PMUT used in IVUS catheters may include a polymer piezoelectric membrane, such as that disclosed in U.S. Pat. No. 6,641,540, hereby incorporated by reference in its entirety. The PMUT transducer can provide greater than 100% bandwidth for optimum resolution in a radial direction, and a spherically-focused aperture for optimum azimuthal and elevation resolution.

The transducer assembly 122 may also include a housing having the PMUT transducer and associated circuitry disposed therein, where the housing has an opening that ultrasound signals generated by the PMUT transducer travel through. In yet another alternative embodiment, the transducer assembly 122 includes an ultrasound transducer array (for example, arrays having 16, 32, 64, or 128 elements are utilized in some embodiments).

The rotation of the imaging core 112 within the sheath 110 is controlled by the interface module 104, which provides user interface controls that can be manipulated by a user. The interface module 104 can receive, analyze, and/or display information received through the imaging core 112. It will be appreciated that any suitable functionality, controls, information processing and analysis, and display can be incorporated into the interface module 104. In an example, the interface module 104 receives data corresponding to ultrasound signals (echoes) detected by the imaging core 112 and forwards the received echo data to the control system 106. In an example, the interface module 104 performs preliminary processing of the echo data prior to transmitting the echo data to the control system 106. The interface module 104 may perform amplification, filtering, and/or aggregating of the echo data. The interface module 104 can also supply high- and low-voltage DC power to support operation of the catheter 102 including the circuitry within the transducer assembly 122.

In some embodiments, wires associated with the IVUS imaging system 100 extend from the control system 106 to the interface module 104 such that signals from the control system 106 can be communicated to the interface module 104 and/or visa versa. In some embodiments, the control system 106 communicates wirelessly with the interface module 104. Similarly, it is understood that, in some embodiments, wires associated with the IVUS imaging system 100 extend from the control system 106 to the monitor 108 such that signals from the control system 106 can be communicated to the monitor 108 and/or vice versa. In some embodiments, the control system 106 communicates wirelessly with the monitor 108.

As discussed above, the transducer assembly 122 includes a miniature ultrasound transducer and associated electronic circuitry. The transducer and the circuitry may be formed separately and later electrically interconnected together. Traditionally, wire bonding may be used to perform such electrical interconnection. However, wire bonding requires expensive wire bonding equipment and specially trained personnel. Wire bonding also requires in most cases a significant amount of thermal input to the bonded substrate, which may result in thermal degradation of the component. Furthermore, wire bonding requires a rather tall “service loop”, which increases the height of the electrical connection and is therefore undesirable.

According to the various aspects of the present disclosure, a micro-structure is used to establish the electrical connections between the miniature ultrasound transducer and the associated electronic circuitry. The various aspects of the micro-structure will now be discussed in more detail below.

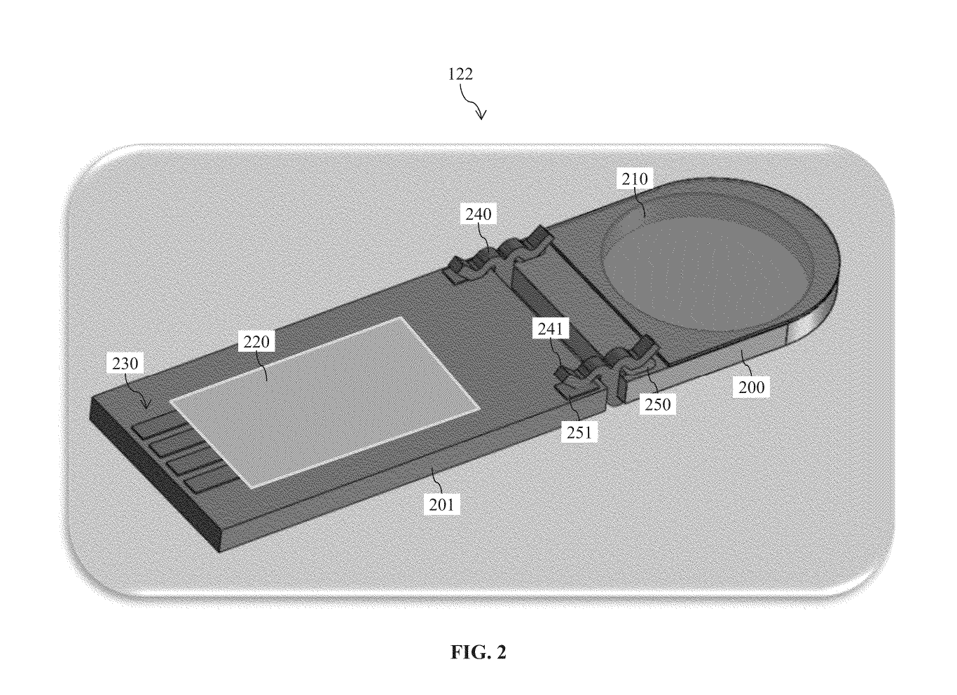

FIG. 2 is a simplified perspective view of a portion of the transducer assembly 122 according to an embodiment of the present disclosure. The transducer assembly 122 includes a micro-component 200 and a micro-component 201. In the illustrated embodiment, the micro-components 200-201 include micro-substrates. These micro-substrates have miniature dimensions, for example they may have a thickness ranging from about 75 microns (um) to about 600 um. In other embodiments, the micro-components 200-201 may include dies or other miniature devices suitable for the growth or placement of microelectronic devices.

An ultrasonic transducer 210 is formed on the micro-component 200. The ultrasonic transducer 210 has a small size and achieves a high resolution, so that it is well suited for intravascular imaging. In some embodiments, the ultrasonic transducer 210 has a size on the order of tens or hundreds of microns, can operate in a frequency range between about 1 mega-Hertz (MHz) to about 135 MHz, and can provide sub 50 micron resolution while providing depth penetration of up to 10 millimeters (mm). Furthermore, the ultrasonic transducer 210 is also shaped in a manner to allow a developer to define a target focus area based on a deflection depth of a transducer aperture, thereby generating an image that is useful for defining vessel morphology, beyond the surface characteristics. In the depicted embodiment, the ultrasound transducer 210 is a piezoelectric micromachined ultrasound transducer (PMUT). In other embodiments, the transducer 200 may include an alternative type of transducer. Additional details of the ultrasonic transducer 210 are described in Provisional U.S. Patent Application 61/745,091 to Dylan Van Hoven, filed on December 21, entitled “Preparation and Application of a Piezoelectric Film for an Ultrasound Transducer”, and attorney docket 44755.1060, and Provisional U.S. Patent Application 61/745,212 to Dylan Van Hoven, filed on December 21, entitled “Method and Apparatus for Focusing Miniature Ultrasound Transducers”, and attorney docket 44755.1061, the contents of each which are herein incorporated by reference in its entirety.

The micro-component 201 contains micro-electronic circuitry for controlling and interacting with the transducer 210. In the illustrated embodiment, such micro-electronic circuitry is implemented as an Application-Specific Integrated Circuit (ASIC) 220, where the micro-component 201 serves as a micro-substrate for the ASIC 220. The ASIC 220 may be electrically coupled to the substrate through conductive pads 230. It is understood that in other embodiments, the micro-component 201 itself may be an Integrated Circuit (IC) chip.

The transducer assembly 122 also includes one or more micro-structures for coupling together the micro-components 200 and 201. In the illustrated embodiment, two such micro-structures are implemented as micro-clips 240 and 241. The micro-clips 240-241 are mostly electrically conductive. It is understood however, that the micro-clips 240-241 may have some insulated areas or may be selectively conductive. The micro-clips 240-241 are clamped down on the upper and lower surfaces of the micro-components 200 and 201. The micro-clips 240-241 may make contact with the micro-components 200 and 201 through corresponding conductive pads, for example conductive pads 250 and 251 that are in physical and electrical contact with the micro-clip 241. Such contact is maintained through either a spring action built in to the micro-clips 240-241 or by crimping the micro-clips 240-241 through an external force. In doing so, the micro-clips 240-241 may establish electrical connections between the ASIC 220 and the transducer 210. According to the various aspects of the present disclosure, the micro-clips 240-241 may be used to electrically interconnect the micro-components 200 and 201 at room temperature. In comparison, wire bonding is associated with extreme heat, which may damage one or more temperature-sensitive devices (such as the transducer 210 or the ASIC 220). A human operator may also perform the assembly using only a microscope and tweezers. Alternatively, automated machines may also be used to perform the assembly.

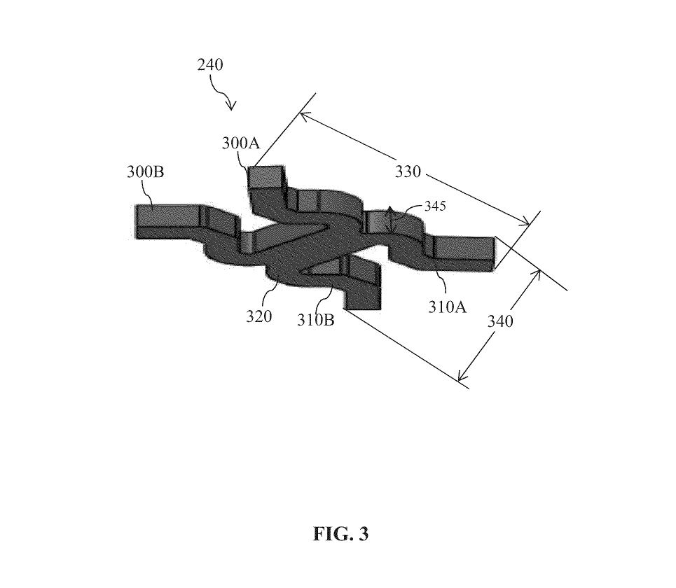

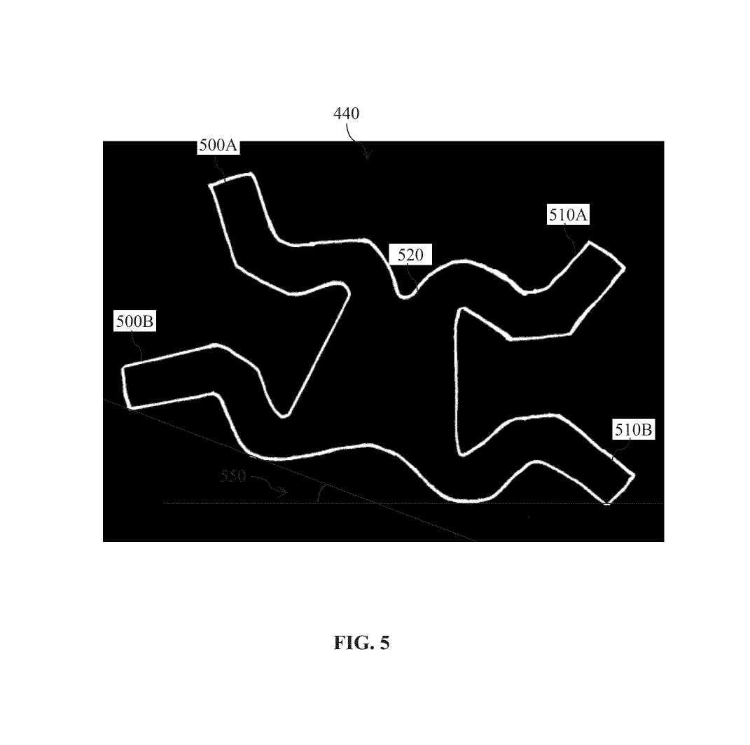

Referring now to FIG. 3, a diagrammatic perspective view of the micro-clip 240 (or the micro-clip 241) is shown in more detail to facilitate the understanding of the concepts disclosed herein. The micro-clip 240 has an arm portion 300 and an arm portion 310 that are joined together by a center portion 320. The arm portion 300 includes a pair of arms 300A and 300B that extends away from the center portion 320 in a first direction. The arm portion 310 includes a pair of arms 310A and 310B that extends away from the center portion 320 in a second direction different from the first direction. In the illustrated embodiment, the first and second directions are opposite one another (i.e., 180 degrees). However, they may not be extending at exactly opposite directions from one another in alternative embodiments, but more at an angle. This will be discussed in more detail below in view of FIG. 5.

The micro-clip 240 is on a miniature scale. For example, a horizontal dimension 330 of the micro-clip 240 is in a range from about 0.2 mm to about 0.4 mm, for example about 0.303 mm, a vertical dimension 340 of the micro-clip 240 is in a range from about 0.15 mm to about 0.21 mm, for example about 0.184 mm, and a thickness 345 of the micro-clip 240 is in a range from about 0.03 mm to about 0.05 mm, for example about 0.04 mm. Of course, the exact numerical values for these dimensions may be different in alternative embodiments.

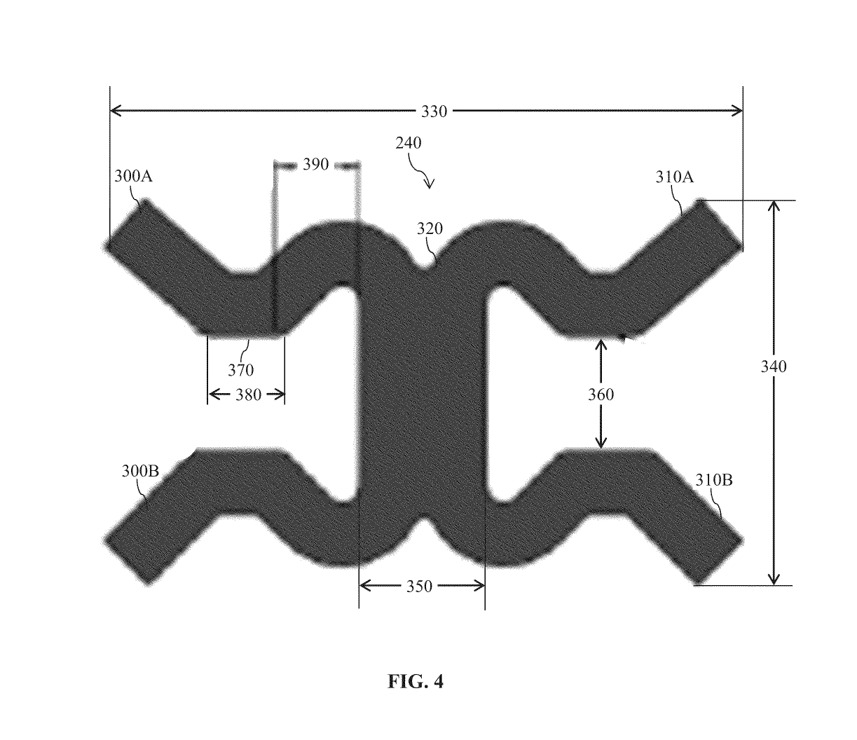

FIG. 4 is a diagrammatic cross-sectional view of the micro-clip 240 (or the micro-clip 241) in more detail. As is shown in FIG. 4, the micro-clip 240 has a “double wishbone” shape, defined by the arms 300A-300B extending in an opposite direction from the arms 310A-310B. The arms 300A-300B and the arms 310A-310B are joined together by the center portion 320. The center portion 320 has a lateral dimension (or width) 350 that defines a minimum distance between the two adjacent micro-components that the micro-clip 240 is configured to electrically interconnect. In some embodiments, the lateral dimension 350 is in a range from about 50 um to about 150 um. Meanwhile, the arm 310A of the micro-clip 240 is separated from the arm 310B by a gap 360, and the same is true for the arms 300A and 300B. The gap 360 is approximately equal to, or less than, a thickness of the micro-component (e.g., the micro-component 200 of FIG. 2) to be clamped down by the arms 310A-310B. In some embodiments, the gap 360 is in a range from about 40 um to about 70 um, for example around 55 um.

In the illustrated embodiment, for each of the arms 300A-300B and 310A-310B, there is a substantially flat surface 370 configured to come into contact with the conductive pads of the micro-components. The relative flatness of the surface 370 facilitates good or sufficient contact between the arms of the micro-clip 240 and the conductive pad of the micro-component. In some embodiments, a lateral dimension 380 of the surface 370 is in a range from about 20 um to about 40 um, for example about 29 um. Also in the illustrated embodiment, a portion of the arm 300A joining the center portion 320 with the portion with the flat surface 370 has a lateral dimension 390. The lateral dimension 390 may be in a range from about 30 um to about 50 um, for example about 40 um.

In some embodiments, the pair of arms 300A-300B is spring-tempered, as are the pair of arms 310A-310B. In other words, a spring force prevents the arms 300A-300B from moving away from one another, and the same is true for the arms 310A-310B. As such, the arms 300A-300B can effectively clamp down on the micro-component that they come into contact with (thereby establishing electrical connection), for example the micro-components 200 or 201. The micro-clip 240 may be made of NiCo in these embodiments.

In other embodiments, the pair of arms 300A-300B is half-hard, as are the pair of arms 310A-310B. In these embodiments, a second step to deform the micro-clip 240 needs to be performed. An external force (applied by an external tool not illustrated herein) may be used to crimp the arms 300A-300B (or the arms 310A-310B) toward each other after these arms have been positioned to align with the contact pads of the micro-component. The deformity caused by the external force once again ensures good electrical contact between the arms (e.g., flat surfaces of the arms) and the conductive pads of the micro-component. The micro-clip 240 may be made of Au in these embodiments. To encourage good electrical connection, the conductive pads of the micro-component also need to be sufficiently thick and soft. For example, the conductive pads may be made of soft Au with a thickness ranging from about 5 to 15 um.

It is understood that while the micro-clips 240-241 provide some degree of mechanical strength between the two micro-components 200-201 (FIG. 2), a secondary part or a suitable adhesive material may also be used to enhance the mechanical strength of the resulting assembly 122. Furthermore, as discussed above, the pair of arms 300A-300B may not be planar with the pair of arms 310A-310B (i.e., not extending in exactly opposite directions).

For example, referring now to FIG. 5, a simplified top view of an alternative embodiment of a micro-clip 440 is shown. Similar to the micro-clip 240 discussed above, the micro-clip 440 has a pair of arms 500A-500B and a pair of arms 510A-510B that are joined together by a center portion 520. The shape or profile of the arms 500A-500B and 510A-510B may be similar to the arms 300A-300B and 310A-310B. However, unlike the arms 300A-300B and 310A-310B, the pair of arms 500A-500B extends at an angle 550 with respect to the pair of arms 510A-510B. This angle 550 allows the micro-components 200 and 201 to be coupled together at an angle. In other words, the micro-components 200-201 would not be co-planar, but rather one of them would be “tilted” if the other one is “flat.” For example, the micro-component 200 with the transducer 210 may be “tilted,” and the micro-component 201 with the ASIC 220 (FIG. 2) may be “flat.” This configuration facilitates Doppler and forward-looking IVUS/FACT applications.

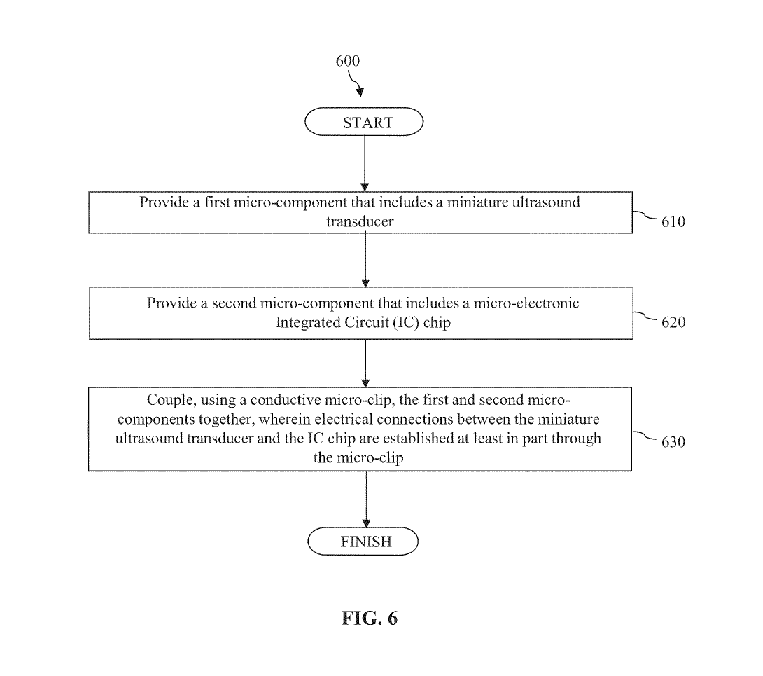

FIG. 6 is a flowchart of a method 600 for fabricating an ultrasonic transducer according to various aspects of the present disclosure. The method 600 includes a step 610, in which a first micro-component is provided. The first micro-component includes a miniature ultrasound transducer. In some embodiments, the miniature ultrasound transducer is formed on a micro-substrate.

The method 600 includes a step 620, in which a second micro-component is provided. The second micro-component includes a micro-electronic Integrated Circuit (IC) chip. In some embodiments, the IC chip is formed on a micro-substrate.

The method 600 includes a step 630, in which the first and second micro-components are coupled together using a micro-clip. The electrical connections between the miniature ultrasound transducer and the IC chip are established at least in part through the micro-clip. The micro-clip includes a first pair of arms and a second pair of arms that are joined together through a center portion. The first pair of arms and the second pair of arms extend in different directions. The step 630 includes clamping down on the first micro-component using the first pair of arms and clamping down on the second micro-component using the second pair of arms.

In some embodiments, the step 630 is performed so that at least one of the first pair of arms and the second pair of arms clamp down on the first or second micro-component through a spring force. In other embodiments, the step 630 includes deforming at least one of the first pair of arms and the second pair of arms such that they clamp down on the first or second micro-component through the deformity. In some embodiments, the first pair of arms, the second pair of arms, and the center portion collectively define a profile that resembles a double wishbone.

It is understood that additional fabrication steps may be performed to complete the fabrication of the transducer. However, these additional fabrication steps are not discussed herein for reasons of simplicity.

Based on the above discussions, it can be seen that the transducer shaping according to the embodiments of the present disclosure offers numerous advantages over conventional methods. Of course, it is understood that not all advantages are necessarily discussed herein, other embodiments may offer different advantages, and no particular advantage is required for all embodiments.

As an example, the micro-clip of the present disclosure obviates the use of wire bonding. The elimination of wire bonding reduces fabrication costs, since wire bonding equipment is expensive and requires special training for its operator(s). Assembling the micro-components together using the micro-clip is also simple, and therefore an operator can easily perform the assembly using simple tools such as a microscope and tweezers. The assembly can also be done in room temperature, whereas the wire bonding would carry the risk of subjecting one or more temperature-sensitive components to extreme heat, which may damage the device. A further advantage of eliminating wire bonding is that the overall height of the transducer assembly may be reduced, since the bonding wires would no longer be present.

Another advantage of the present disclosure is that the micro-clip enhances the strength of the coupling between the micro-components. The rotational and axial positioning of the transducer assembly is maintained as a result of the arms of the micro-clip clamping down on the micro-components.

Yet another advantage of the present disclosure is that the micro-clip can be configured to allow for non-planar orientation of the coupled micro-components. For example, the arms of the micro-clip can be configured to extend at an angle with respect to one another (e.g., FIG. 5). This allows the micro-components (as clamped down) to also be oriented at an angle with respect to each other. As discussed above, this angular positioning in beneficial in applications involving Doppler and forward-looking IVUS/FACT devices.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A micro-structure for coupling a first micro-component with a second micro-component, the micro-structure comprising:
a center portion;
a first pair of arms connected to the center portion and extending away from the center portion in a first direction, the first pair of arms being separated by a first gap that is approximately equal to, or less than, a thickness of the first micro-component; and
a second pair of arms connected to the center portion and extending away from the center portion in a second direction different from the first direction, the second pair of arms being separated by a second gap that is approximately equal to, or less than, a thickness of the second micro-component.

2. The micro-structure of claim 1, wherein:
the first and second pairs of arms each include two substantially flat surfaces facing toward each other;
the two substantially flat surfaces of the first pair of arms are configured to make contact with an upper surface and a lower surface of the first micro-component; and
the two substantially flat surfaces of the second pair of arms are configured to make contact with an upper surface and a lower surface of the second micro-component.

3. The micro-structure of claim 1, wherein the micro-structure contains electrically conductive portions and electrically insulated portions.

4. The micro-structure of claim 1, wherein at least one of the first pair of arms and the second pair of arms are spring-tempered.

5. The micro-structure of claim 4, wherein the micro-structure includes NiCo.

6. The micro-structure of claim 1, wherein at least one of the first pair of arms and the second pair of arms are half-hard.

7. The micro-structure of claim 6, wherein the micro-structure includes Au.

8. The micro-structure of claim 1, wherein the micro-structure has a double wishbone shape.

9. The micro-structure of claim 1, wherein the first micro-component and the second micro-component each include a micro-substrate.

10. The micro-structure of claim 1, wherein the center portion includes a dimension defining a minimum distance between the first and second micro-components.

11. The micro-structure of claim 1, wherein the second direction is opposite the first direction.

12. An assembly for an ultrasound imaging device, the assembly comprising:
a first micro-component having an upper surface and a lower surface, wherein the first micro-component includes a miniature ultrasound transducer;
a second micro-component having an upper surface and a lower surface, wherein the second micro-component includes a micro-electronic Integrated Circuit (IC) chip; and
a micro-clip coupled to the first and second micro-components, wherein the micro-clip includes a first pair of arms clamped to the upper and lower surfaces of the first micro-component and a second pair of arms clamped to the upper and lower surfaces of the second micro-component.

13. The assembly of claim 12, wherein the micro-clip has a double wishbone shape.

14. The assembly of claim 12, wherein the micro-clip includes:
a first pair of arms being separated by a first gap that defines a thickness of the first micro-component; and
a second pair of arms being separated by a second gap that defines a thickness of the second micro-component.

15. The assembly of claim 14, wherein at least one of the first pair of arms and the second pair of arms are configured to clamp down to the first or second micro-component through a spring force.

16. The assembly of claim 14, wherein the first pair of arms and the second pair of arms are joined together by a center portion, and wherein the center portion includes a lateral dimension defining a minimum distance between the first and second micro-components.

17. The assembly of claim 12, wherein the micro-clip includes electrically conductive portions and electrically insulated portions.

18. The assembly of claim 12, wherein the micro-clip establishes electrical contact between the first and second micro-components and maintains rotational and axial positioning.

19. The assembly of claim 12, wherein the first micro-component and the second micro-component each include a micro-substrate.

20. A method of fabricating a miniature ultrasound transducer, the method comprising:
providing a first micro-component that includes a miniature ultrasound transducer;
providing a second micro-component that includes a micro-electronic Integrated Circuit (IC) chip; and
coupling, using an at least partially conductive micro-clip, the first and second micro-components together, wherein electrical connections between the miniature ultrasound transducer and the IC chip are established at least in part through the micro-clip.

21. The method of claim 20, wherein the micro-clip includes a first pair of arms and a second pair of arms joined together through a center portion, the first pair of arms and the second pair of arms extending in different directions.

22. The method of claim 21, wherein the coupling comprises clamping down on the first micro-component using the first pair of arms and clamping down on the second micro-component using the second pair of arms.

23. The method of claim 21, wherein the first pair of arms, the second pair of arms, and the center portion collectively define a profile that resembles a double wishbone.

24. The method of claim 21, wherein the coupling is performed so that at least one of the first pair of arms and the second pair of arms clamp down on the first or second micro-component through a spring force.

25. The method of claim 21, wherein the coupling comprises deforming at least one of the first pair of arms and the second pair of arms such that they clamp down on the first or second micro-component through the deformity.

26. The method of claim 20, wherein the providing the first micro-component and the providing the second micro-component are performed such that they each include a micro-substrate on which the micro-clip is clamped.

27. The method of claim 20, wherein the micro-clips includes electrically conductive portions and electrically insulated portions.
